(19) United States
(12) Patent Application Publication
(10) Pub. No.: US 2009/0223524 A1
(43) Pub. Date: Sep. 10, 2009

(54) Leg and feet elevator

(76) Inventor: Thomas W. Daivs, III, (US)

Correspondence Address: Thomas Walter Davis III, 10879 Woodpine Trl., Bethanl, LA, 71007, US

(21) Appl. No.: 12/380,694

(22) Filed: Mar. 3, 2009

Related U.S. Application Data
(60) Provisional application No. 61/068,077, filed on Mar. 4, 2008.

(51) Int. Cl. A61F 5/00

(52) U.S. Cl. 128/845

(57) ABSTRACT

This lounging device is a leg and feet elevator apparatus that should be used for recreational and/or therapeutic purposes. The device is triangular in shape with a right angle design for optimum comfort and multi-positioning; wrapped in an outer cover of durable vinyl/plastic; inflatable composite material and heat sealed envelopes that give the device its wedge effect for comfort and support of the legs and feet. On either side of the center surface are air diffusers which allow air to diffuse from the center surface to comfort and support the legs and feet. The elevating wedge operates to elevate the legs and feet at an angle comfortable for lounging in a semi-upright position for many recreational and therapeutic purposes. This is a cost efficient device that everyone can afford. Other embodiments are described and shown.

### CROSS-REFERENCE TO RELATED APPLICATIONS

The present Utility patent application claims priority benefit of the U.S. provisional application for patent U.S. 61/068,077, entitled “Leg and Feet Elevator” and filed on Mar. 4, 2008 under 35 U.S.C. 119(c) by the present inventor. This device relates to lounging devices and more specifically to such devices which are suitable for recreational and therapeutic needs. The contents of this related non-provisional application are incorporated herein by reference.

### FIELD OF THE DEVICE

The present device relates generally to recreational and therapeutic lounging. More particularly the device relates to an indoor/outdoor lower body elevation device that is specifically suitable for anyone.

### BACKGROUND OF THE DEVICE

In most cases people lounge for either recreation or therapy, whichever the case may be proper lower body support is necessary. Consumers need to elevate their lower extremities for recreational and therapeutic needs when lounging both indoors and outdoors. Lounging in a semi-upright position without proper leg and foot support can cause various physical strains. It is accordingly an object of the present device to greatly increase the comfort of the consumer while adding light-weight and portable features that will result in the multi-positioning use of this device. Known devices for positioning include but are not limited to variations of supportive pillows of soft or in some cases beaded filling or memory foam wedges that do not provide the firm support surface, mobility, cost-efficiency and durability of this particular device.

Some known devices are larger foam wedges providing only one angle of elevation without the ability to move easily into different areas. There are wedges that are used strictly in medical professions for supporting the lower extremities or inclined mattresses—which are too expensive for the average consumer. This device has multiple positioning for the consumer due to its right angle triangle construction. The support of the leg and feet elevator relieves strain from the lower extremities when left hanging downward from lounging in a semi-upright position. Lounging in a semi-upright position can be less restrictive, comfortable, and even prevent poor blood circulation to the legs when lying flat if the Leg and Feet Elevator is applied.

Yet other known devices for lounging are recommended for use in one area or another, they are not portable and cannot be taken with you. What is commonly used in the home to position consumers in a lounging position are pillows and rolled up blankets to prop up the consumers leg and feet on flat or elevated mattresses or in non-adjustable seats.

In view of the foregoing, when in a semi-upright or flat position a device is needed for the support and comfortable positioning of the consumers leg and feet for recreational and therapeutic purposes. A device is also needed for the positioning of a consumer's leg and feet that will alleviate poor circulation, strains and pains of lounging in uncomfortable, unsupportive, and unstable positions. This device is safe, portable, easy to use, light-weight and effective for therapeutic needs. It would further be desirable if the device also provides safe, easy, effective, accessible and comfortable positioning of the consumer's lower body, to facilitate effective air exchange and support for the leg and feet

### BRIEF DESCRIPTION OF THE DRAWINGS

The present device is illustrated by way of example, and not by way of limitation, in the figures of the accompanying drawings and in which like reference numerals refer to similar elements:

FIG. 1 shows a landscape/side view of the “Leg and Feet Elevator”, and various components thereof;

FIG. 2 shows a view from X-axis (above) of said embodiment. To give one a better understanding of said device;

FIG. 3 shows a side view with cutaway section, showing the inner construction and parts;

FIG. 4 shows a cutaway cross-section view at the apex;

FIG. 5 shows a different construction configuration embodiment of the present device;

FIG. 6 shows an illustration of “FIG. 5” cross-section cutaway section and said components;

Unless otherwise indicated illustrations in the figures are not necessarily drawn to scale.

### SUMMARY OF THE DEVICE

To achieve the forgoing and other objects and in accordance with the purpose of the device, an apparatus for a “Leg and Feet Elevator” is described.

In one embodiment, a Leg and Feet Elevator apparatus having an elevating wedge, pneumatic cushions and air diffusers is designed to provide recreational and therapeutic lounging operable to support the legs and feet at a comfortable angle. The design of this device has been structured at a right angle triangle to allow multi-positioning for both leg and feet support. The outer cover is shown comprising heat-sealed inner and side panels to support the device. Indicator marks are formed to show bends in the surface to contour the consumer's body parts for maximum comfort. Air diffusers are in the side panel for an even flow of support.

This embodiment is designed as an inflatable, portable, multi-use, elevating wedge. This embodiment is also cost efficient.

Other features, advantages, and objects of the present device will become more apparent and be more readily understood from the following detailed description, which should be read in conjunction with the accompanying drawings.

### DETAILED DESCRIPTION OF THE PREFERRED EMBODIMENTS

The present device is best understood by reference to the detailed figures and description set forth herein.

Embodiments of the device are discussed below with reference to the Figures. However, those skilled in the art will readily appreciate that the detailed description given herein with respect to these figures is for explanatory purposes as the device extends beyond these limited embodiments.

A portable leg and feet elevator will be described in which the preferred embodiment combines a pneumatic cushion and hospital wedge designed at a right angle triangle for multi-positioning. It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details. It is to be further understood that while many design features are described below in the context of complying with pertinent government regulations, alternative embodiments and applications of the present may include corresponding design features that may be only partially or not at all in compliance, such non-compliant embodiments and applications of the present device are none-the-less within the scope of the present device. Likewise, all safety oriented design features are optional, and may be replaced with any suitable implementation approach known to those skilled in the art.

It is contemplated that other embodiments of the present device may implement other suitable designs, depending upon the needs of the particular consumer, for example, without limitation, the base could be sloped, however this embodiment places too much support on the lower extremities. This device is more suitable for anyone who likes lounging but prefers to do so in a semi-upright position with support for the legs and feet. This device is to be used for recreational and/or therapeutic purposes for older children up to adult and is not suitable to be used as a flotation device.

The entire device is portable and an outer covering wraps completely around the device to help prevent skin from sweating, sliding, and accidental punctures or tears to the cushions. The device may be placed on the floor inside, outside, on the ground—concrete, grass or dirt. The measurements provided are for the preferred embodiment of the present device, and those skilled in the art, in light of the present teachings, will recognize that alternate measurements may also be suitable for a Leg and Feet Elevator.

The present embodiment is comprised of a type of plastic/rubberized plastic, vinyl, light-weight material suitable for inflation. This material can be made from a flame retardant, waterproof, non-breathable material that is mildew proof, or covered with nonflammable, waterproof material. The device is preferably flame retardant, and meets pertinent government regulations; such as, without limitation, the NFPA 701 Flame Resistance and MIL-F-21840F shear strength requirements. The vinyl portions are preferably waterproof, tear resistant, rot resistant, and are in compliance with pertinent government regulations; such as, without limitation, NFPA 701 Flame Resistance and State of Louisiana F-222.03 Flame Retardant Rating.

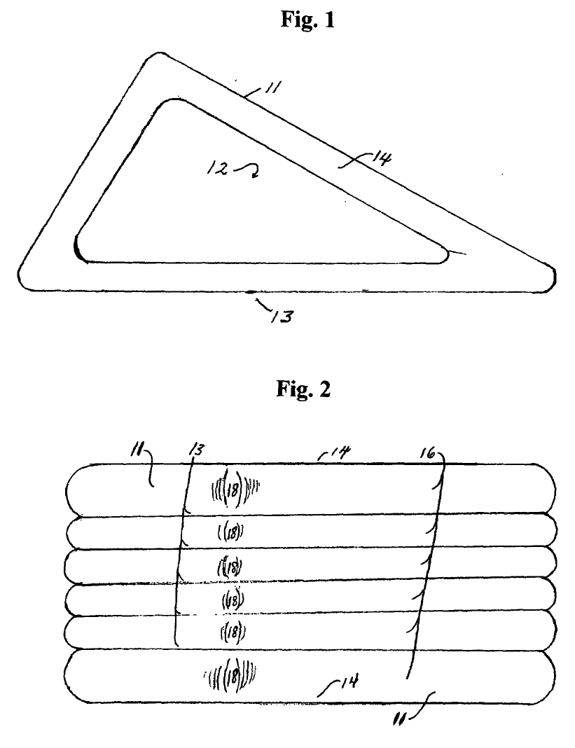

FIG. 1 shows a landscape/side view of the “Leg and Feet Elevator”, and various components thereof: Pneumatic cushions are used to design the present embodiment in an elevation of approximately 30×60×90 degrees in measurement. This right angle triangle design is created for support of the lower extremities and can be used for any recreational or therapeutic activity. The side panel 12 is connected to the rolled edge 14 of each side of the device to maintain the devices stability with respect to the heat sealed 13 rolled edge 14 and the outer cover 11 which conforms the devices shape because it wraps completely around the device and is provided for support and comfort to skin, reduction in sliding and easy care. The elevator of the present embodiment has an angle for comfort and prevention of pressure to the legs and feet. This embodiment is also more affordable than the aforementioned.

FIG. 2 shows a view from the X-axis, (above) of said embodiment. To give a better understanding of said device there are several envelopes 16 formed between panels for necessary support of the lower extremities. Each envelope 16 is heat sealed 13 and encompassed by the rolled edge 14 for added support. At the apex of this device are indicator marks 18 to illustrate bends in the surface for a more contoured shape. The outer cover 11 wraps completely around the device and is constructed of a non-porous, vinyl/plastic, flame retardant material.

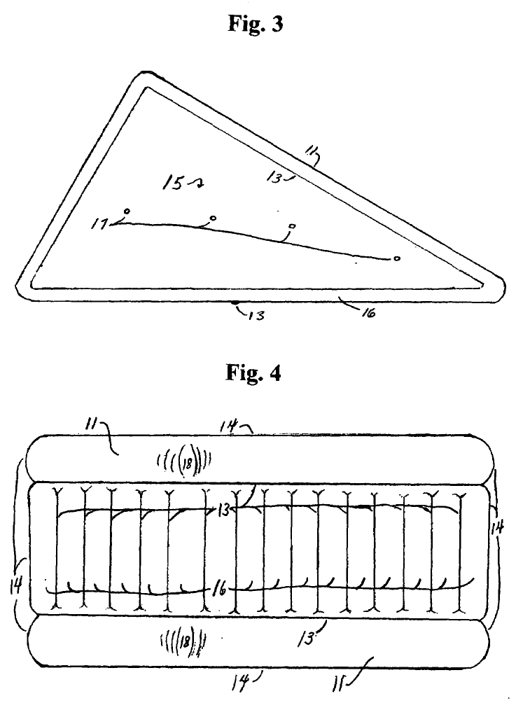

FIG. 3 illustrates a side view with cutaway section showing the inner construction and parts; a view of the components of the elevating apparatus of an exemplary leg and feet elevator, in accordance with an embodiment of the present device. The inner panels 15 are equipped with air diffuser ports 17 which allow air to flow to and from the heat sealed 13 envelopes 16; the outer cover 11 is heat-sealed 13 as well for proper support for lounging comfortably.

FIG. 4 shows a cutaway cross-section view at the apex; the heat sealed 13 envelopes 16 that are formed between panels are encompassed by a rolled edge 14 on either end of the device. Indicator marks 18 at the apex illustrate bends in the surface. The outer cover 11 which is a waterproof, nonporous, mildew resistant material wraps around the device completely.

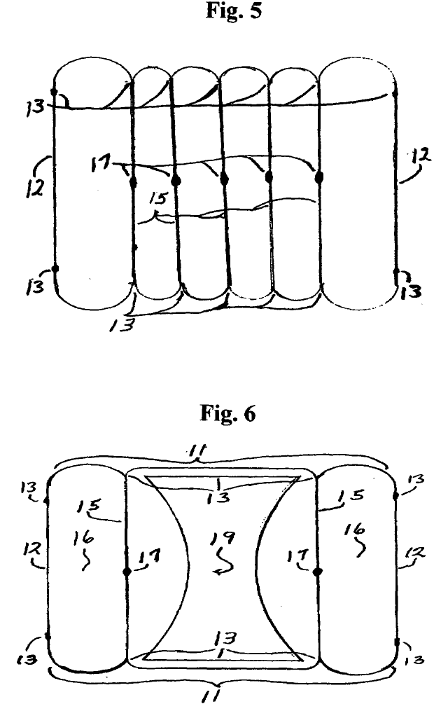

FIG. 5 shows a different construction configuration embodiment of the same device with a transverse or crossways tubular configuration. This view shows the heat sealed 13 inner panels 15 with air diffuser ports 17 between each inner panel. The heat sealed 13 side panels 12 encompass the inner panels 15 for added support.

FIG. 6 shows FIG. 5 cross-section cutaway section and said component parts. Heat sealed 13 inner panels 19 are surrounded by air diffuser ports 17 which allow air to flow to and from the inner panels 15. The envelopes 16 formed between the panels are connected to the heat sealed 13 side panel 12 which are encompassed by the outer cover 11 that wraps completely around the right triangular shaped device.

The foregoing embodiments of the present device comprise a multiplicity of aspects, which, for the sake of clarity, are next highlighted separately by way of example and not limitation. For example, one aspect of the preferred embodiment of the present device is to provide elevation of the consumer's leg and feet with the use of an approximately 20-40 degree range to allow for comfortable, effective, versatile positioning of the leg and feet.

Another aspect of the preferred embodiment of the present device is that it can be easily cleaned and meets most, if not all, sanitation needs for its repeated use. Another aspect of the preferred embodiment of the present device is that it is constructed of durable and waterproof materials.

Embodiments of the present device may be used in the home or outside the home for use by anyone who participates in recreational or therapeutic lounging.

Alternative embodiments can not address cost reduction or regulatory issues for certain applications like this one.

Having fully described at least one embodiment of the present device, other equivalent or alternative methods of implementing an leg and feet elevator according to the present device will be apparent to those skilled in the art. The device has been described above by way of example, and the specific embodiments disclosed are not intended to limit the device to the particular forms disclosed. This device is thus to cover all modifications, equivalents, and alternatives falling within the spirit and scope of the following claims.

### Legend

### Drawings—Figures

In the drawings, closely related figures have the same numbers but different alphabetic suffixes.

FIG. 1 shows a landscape/side view of the “Leg and Feet Elevator” and various components thereof.

FIG. 2 shows a view from X-axis, (above) of said embodiment. To give one a better understanding of said device.

FIG. 3 shows a side view with cutaway sections, showing the inner construction and parts.

FIG. 4 shows a cutaway cross-section view at the apex.

FIG. 5 shows a different construction configuration embodiment of the same device.

FIG. 6 shows FIG. 5, cross-section cutaway section and said component parts.

### Drawings—Reference Numerals

11 outer cover (wrap completely around)
12 Side panel
13 heat seal
14 rolled edge
15 inner panels
16 envelopes (formed between panels)
17 air diffuser ports
18 indicator marks (bends in surface)
19 inner panels (see FIG. 5)

1. A leg and feet elevator apparatus comprising: leg, joint and knee support; said support comprising a plastic/vinyl apparatus. The innermost inner panel is encompassed by the envelopes that are formed between the panels to create the support needed for this device; air diffuser ports are on the inner side panels to allow air to flow to and from the inner panel for even inflation and deflation; side panels and rolled edges are connected to the heat sealed envelopes for added stability; outer cover wraps completely around device for durability, to prevent sliding and easy care; indicator marks are illustrated to show bends in the surface at the apex of the device;

2. Said elevating wedge operable to elevate said lower extremities at an angle comfortable for recreation and therapeutic use for everyone.
a. The apparatus as recited in claim 1 and 2, in which said device is designed at a right angle triangle.
b. The apparatus as recited in claim 1 and 2, further comprising elevating heat sealed envelopes in the center by inner panel.
c. The apparatus as recited in claim 1 and 2, in which said device is multi-positioning for the consumer.
d. The apparatus as recited in claim 1 and 2, in which said device is constructed from a pneumatic cushion type material.
e. The apparatus as recited in claim 1 and 2, further comprises said rolled edge and side panels for stability
f. The apparatus as recited in claim 1 and 2, further comprises an outer cover that wraps completely around device for easy care, durability and mobility.
g. The apparatus as recited in claim 1 and 2, further comprises a leg and feet elevation apparatus comprising: a lounging means for anyone to comfortably lounge in a semi-upright position with support for the legs and feet; and an elevating means for adjusting an inclination of said lounging means.
h. The apparatus as recited in claim 1 and 2, further comprising: a support means for supporting a person with leg and feet injuries.
i. The apparatus as recited in claim 22, further comprising: a positioning means for facilitating proper body alignment.
j. The apparatus as recited in claim 1 and 2, further comprising: a securing means for support for therapeutic uses.
k. The apparatus as recited in claim 1 and 2, further comprising: a means for providing support for comfortable recreational purposes.
l. The apparatus as recited in claim 1 and 2, further comprising: a means for securing said semi-upright leg and feet support as a portable structure.
